(19) United States
(12) Patent Application Publication
(10) Pub. No.: US 2008/0075143 A1
(43) Pub. Date: Mar. 27, 2008

(54) DIGITAL THERMOMETERS WITH AUDIBLE FEEDBACK

(76) Inventors: Vanessa Lampke-Honeyghan, Cranston, RI (US); Charles W. Miga, East Greenwich, RI (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/533,927

(22) Filed: Sep. 21, 2006

(51) Int. Cl. G01K 7/00; G01K 1/00; A61B 5/00; G08B 17/00

(52) U.S. Cl. 374/164; 340/584; 374/208; 600/549

(57) ABSTRACT

A digital thermometer is provided that gives audible feedback when the thermometer is properly positioned and can give audible directions to reposition the thermometer and start the process again when the thermometer ceases to be properly positioned. A probe has a temperature sensor at a first end adapted to be inserted into the mouth and under the tongue, and a housing portion at the second end. A display component displays the measured temperature. An audio component plays music while the probe is properly positioned and issues a command to try again when the probe ceases to be properly positioned. Electronic circuitry receives a temperature signal from the temperature sensor, translates the signal into a value representative of the measured temperature, and provides the translated value to the display for displaying the temperature. Electronic circuitry also detects improper positioning of the probe by detecting a rapid decrease in temperature at the temperature sensing end.

### BACKGROUND OF THE INVENTION

This invention relates generally to apparatus for electronically measuring the temperature of a living body, and more particularly to a digital thermometer that indicates whether the thermometer is properly positioned to take an accurate reading and provides audible feedback during operation of the thermometer to assist in indicating that the thermometer is properly and/or improperly positioned within the mouth of the subject.

Electronic thermometers have been introduced which emulate the structure of the basic mercury thermometer. A numerical display is usually disposed on electronic thermometers to assist in reading the measured temperature. These electronic thermometers have a temperature sensing tip at one end for insertion under the tongue, an intermediate shaft and an enlarged body or housing at the end opposite from the sensing tip. The enlarged housing contains electronics for translating a temperature signal from the sensing tip into the currently measured temperature. Typically, the enlarged housing also contains a display, such as of the liquid crystal display (LCD) type, for displaying the measured temperature. The enlarged housing also contains a source of electrical power, such as a miniature battery.

Such electronic thermometers with digital displays offer improved convenience and accuracy compared to reading the linear scale of the basic mercury thermometer. Such electronic thermometers may also provide an audible beep to indicate various phases of the temperature measuring process However, these electronic thermometers do not provide appropriate feedback to patients who are not accustomed to, or are uncooperative in, having their temperature taken, such as children.

### SUMMARY OF THE INVENTION

The present invention provides a digital thermometer with the capability to detect when the temperature sensing probe is either properly or improperly positioned during operation. Improper positioning can be indicated by a decrease in temperature after a temperature rise is detected. Proper positioning can be indicated by having the digital thermometer give audible feedback such as music during operation until improper positioning is indicated, typically by a detected temperature drop, especially after a rising temperature had been detected. In an embodiment, the digital thermometer gives audible feedback such as cessation of music and/or a verbal command to try again when the probe detects a decrease in temperature indicating that the temperature sensing probe may not be properly positioned during operation.

A probe of the electronic digital thermometer is disposed between a sensing tip at one end and a housing at the opposite end. A temperature sensing end of the probe near the temperature-sensing tip is disposed to be positioned under the tongue of a patient and in contact with the floor of the mouth of the patient. The housing contains a display, a power source such as a battery, an audible device such as a speaker, and control circuitry.

In its broadest form, a digital thermometer having characteristics of the invention may be a probe adapted to be inserted into the mouth. The probe could be connected to a housing portion, or it could be connected by electrical conductors to a remote box with a display.

It is a general aspect or object of the present invention to provide a digital thermometer and method for measuring the temperature of a living being with appropriate feedback to signal compliance with proper positioning of the temperature sensing tip, such as under the tongue of a patient, in order to promote enhanced accuracy.

Another aspect or object of the present invention is to provide a digital thermometer indicating the thermometer likely is not properly positioned while measuring the temperature of a human being such as by providing appropriate audible feedback when the temperature sensing tip detects a decreasing temperature.

Another aspect or object of the present invention is to provide a digital thermometer for measuring the temperature of a human being with appropriate audible directions to properly reposition the temperature sensing probe after it has detected a decreasing temperature.

Yet another aspect or object of the present invention is to provide a digital thermometer with the ability to detect when the temperature sensing probe ceases to be in compliance with positioning guidelines.

These and other aspects, objects, features and advantages of the present invention, including the various features used in various combinations, will be better understood in connection with the following drawings in which certain specific features are shown and description of the preferred embodiments

### BRIEF DESCRIPTION OF THE DRAWINGS

FIG. 1 is an elevational view of an embodiment of a digital thermometer incorporating features of the present invention;

FIG. 2 is a cross-sectional side view of the interior of the digital thermometer of FIG. 1;

FIG. 3 is a perspective view of another embodiment of a digital thermometer incorporating features of the present invention;

FIG. 4 is a perspective view of another embodiment of a digital thermometer incorporating features of the present invention;

FIG. 5 is an elevational view of yet another embodiment of a digital thermometer incorporating features of the present invention;

FIG. 6 is a side view illustration of the anatomy in and around a human mouth with the probe portion of the digital thermometer inserted in the mouth; and

FIG. 7 is flow chart illustration of the steps that an embodiment of the digital thermometer executes when in the operational mode to measure temperature.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

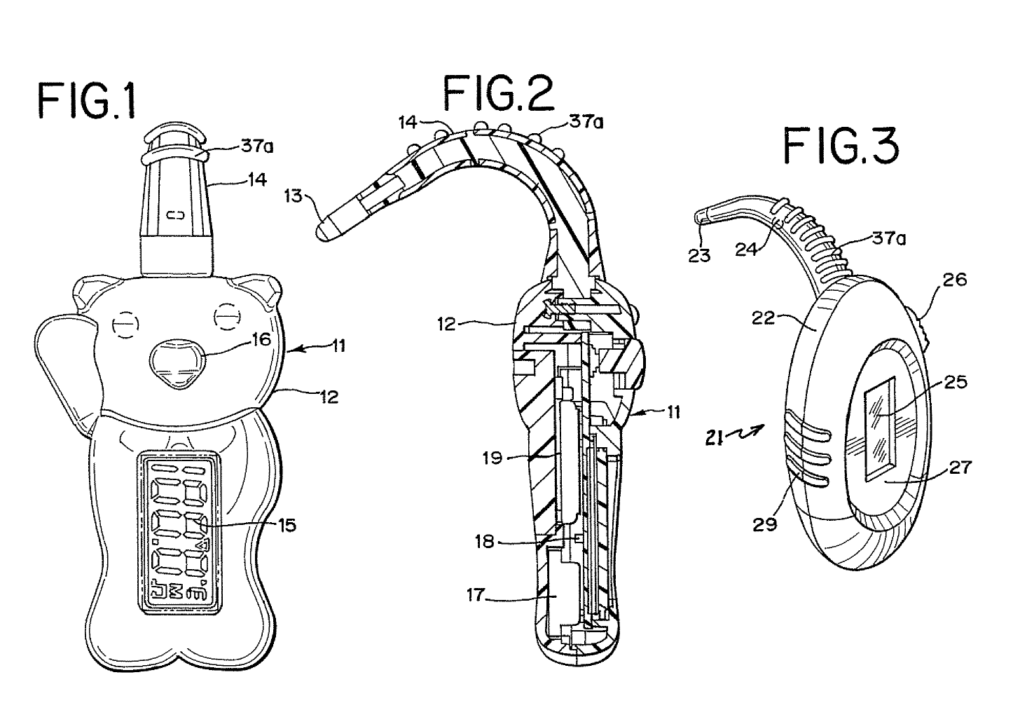

The various drawing figures will now be referred to in detail. Turning first to FIG. 1, a digital thermometer, which is especially suitable for measuring the body temperature of a human being by taking a temperature measurement in the mouth, is generally designated 11. The illustrated digital thermometer 11 includes a housing 12, a probe 14 and a display 15 in the housing 12. A switch 16 is manually operable to electrically activate or deactivate the thermometer 11. For example, switch 16 may be of the pushbutton type.

FIG. 2 illustrates some of the interior details of the thermometer 11. A temperature sensing tip 13 is at the end of probe 14 projecting from the housing 12. The housing 12 also contains electronic circuitry 18, a speaker 17 and a battery 19.

FIG. 3 illustrates another embodiment of a digital thermometer, disclosed in U.S. Pat. No. 7,060,037, which is hereby incorporated by reference. The illustrated digital thermometer, generally designated 21, includes a housing 22, a temperature sensing tip 23, a probe 24 and a display 25 in the housing 22. A switch 26 is manually operable to electrically activate or deactivate the thermometer 21. The housing 22 also contains electronic circuitry, a speaker and a battery.

Grip members 37a are provided to improve the grip of the thermometer 21 with respect to the mouth area and teeth. These are shown as raised ribs; alternatively the grip members could take the form of recessed grooves or otherwise exhibit uneven or gripping characteristics. A plurality of grip members are illustrated, such being oriented on the upper side of the curved portion of the probe. In addition, raised ribs 29 or other suitable gripping member or members are provided for assisting in holding the thermometer during use.

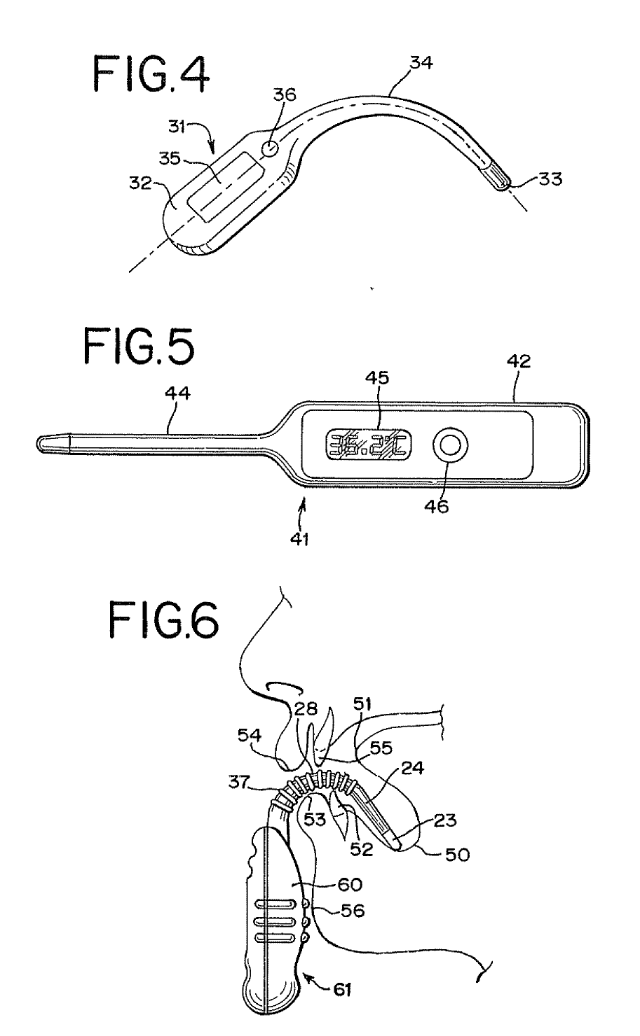

FIG. 4 illustrates yet another embodiment of a digital thermometer, disclosed in U.S. Pat. No. 7,052,471, which is hereby incorporated by reference. The illustrated digital thermometer, generally designated 31, includes a housing 32, a temperature sensing tip 33, a probe 34 and a display 35 in the housing 22. A switch 36 is manually operable to electrically activate or deactivate the thermometer 31. The housing 32 also contains electronic circuitry, a speaker and a battery.

FIG. 5 illustrates a further embodiment of a digital thermometer, generally designated 41. Thermometer 41 includes a housing 42, a temperature sensing tip 43, a probe 44 and a display 45 in the housing 42. A switch 46 is manually operable to electrically activate or deactivate the thermometer 41. The housing 42 also contains electronic circuitry, a speaker and a battery. Further details of this embodiment can be found in U.S. Pat. No. 5,626,425, which is hereby incorporated by reference.

FIG. 6 illustrates another embodiment of a digital thermometer, generally designated 61. It is known in the medical arts that oral measurement of body temperature is most effective if the temperature sensor is in contact with tissues at the floor 50 of the mouth under the tongue 51. It is also known that two “hot spots” exist under the tongue along the inside of the jaw to either side of the base of the underside of tongue 51. Ideally, a thermometer probe is positioned at one or more of these hot spots during use.

The fit of the digital thermometer 61 (and other embodiments thereof) to the anatomy in and around the mouth can now be better appreciated. The probe 24 rests on the lower lip 53 and/or the lower teeth 52. When provided, the grip members, shown as circumferential ribs 37 in this particular embodiment, help to maintain this positioning. That portion of probe 24 resting on lip 53 and/or teeth 52 is an intermediate portion 28 which is shaped to accommodate and/or generally conform to the shape of the lower lip 53 and/or lower teeth 52. This non-linear or curved contour of the probe 24, especially in conjunction with grip members when provided, effectively prevents movement or shifting of the probe 24 while the temperature measurement is taking place. Because thermometer 11, 21, 31, 41 or 61, for example, does not become dislodged from under the tongue 51, either of these thermometers can more quickly reach a final measured temperature than prior art thermometers.

It can be seen that the attachment of the probe to the housing in FIG. 6 is at an inclined angle. In this illustrated embodiment, this contour is of a curved intermediate portion 28. Smooth and constant contours need not be the limit of suitable contours, which can accommodate the lower lip and lower teeth by means of other protruding offset lengths which extend more upwardly (as viewed in FIG. 6) than the rest of the probe 24 or housing 22. The illustrated curved contour achieves these objectives of the invention in an especially efficient and effective manner, which is relatively straightforward to manufacture. The previously described ribs 37, 37a on probe 24 when provided impart additional friction against the lower lip and/or lower teeth that assists in keeping the probe in place.

At the same time, the rear housing portion 30 of thermometer 21 is generally downwardly disposed resting near or against the chin 56 of the patient. Thus, any biting down or pressure on probe 24 with the upper lip 54 and/or upper teeth 55 will not cause the sensing tip 23 of probe 24 to rotate out of position since any significant rotation is prevented when there is contact of rear housing portion 30 against the chin 56. Because the digital thermometer 21 better conforms to the anatomy of the mouth, thermometer 21 also feels less intrusive to a patient than the prior art linear thermometers.

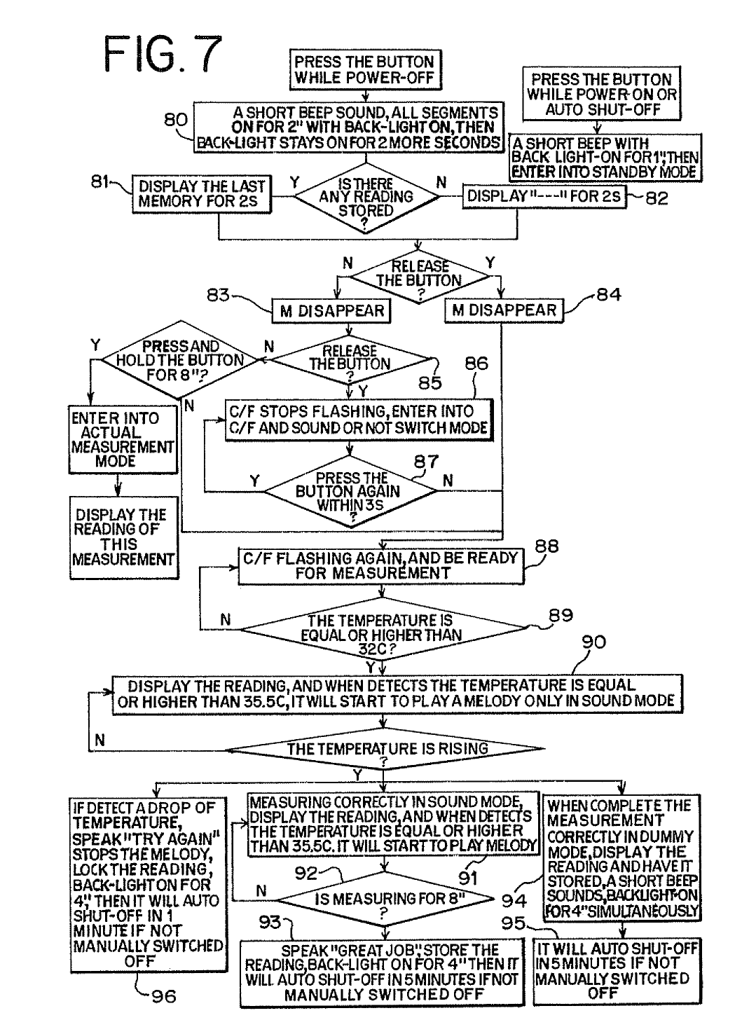

FIG. 7 is a flowchart which illustrates the steps that can be employed by any of the digital thermometers 11, 21, 31 or 41 in measuring the temperature of a patient. For clarity and for illustrative purposes, the following reference numbers correspond to digital thermometer 11. It is to be understood that the following flowchart description applies to the corresponding reference numbers and components of digital thermometers 21, 31, 41 and 61 as well

When the power to thermometer 11 is first turned on by depressing and releasing the power switch 16, all of the indicia and the backlight of display 15 activate for about 2 seconds as indicated by block 80 The backlight then stays on for about another 2 seconds as indicated by block 80. The thermometer also may emit one or more audible beeps as indicated by block 80. This indicates to the user that thermometer 11 is responding to switch 16 and that the internal battery, or other power source, is at a suitable operating voltage.

Digital thermometer 11 then displays the last measured temperature for a short time period, such as about two seconds, as indicated at block 81 If there is no last temperature reading stored, digital thermometer 11 displays a suitable indicia, such as several dashed lines “---”, for about two seconds as indicated at block 82. It is desirable to save the last measured temperature since medical personnel may not have recorded the measurement, or may have pressed switch 16 to turn the thermometer off before recording the temperature By saving and displaying this information, thermometer 11 reminds the user of the prior measurement, which can be designated by an “M” symbol. After the last temperature reading or indicia such as dashed lines are displayed for a short time period such as about two seconds, the “M” symbol (if present) disappears as shown at block 84, and thermometer 11 is ready to begin temperature measurement as shown at block 88. The default operation for thermometer 11 is “F/Melody” for degrees Fahrenheit and music on.

If power switch 16 is held down for a relatively longer time period at startup, such as about four seconds, the “M” symbol disappears as shown at block 83. If power switch 16 is then released, the thermometer enters the switchable mode function shown at block 86. If the power switch 16 is then pressed and released within about two seconds, the mode will switch to “F/Quiet” for degrees Fahrenheit and no music. Successive similar activations within about two seconds of power switch 16 will change the mode to “C/Quiet” for degrees Celsius and no music; then to “C/Melody” for degrees Celsius and music on; and then back to “F/Melody” as shown in decision block 87.

Thermometer 11 then waits for another interval, such as about another two seconds, to make sure that the appropriate measurement scale and sound mode have been selected. Thermometer 11 is then ready to begin temperature measurement as shown in block 88.

Thermometer 11 then proceeds to decision block 89 where the first new measurement is made. If the measured temperature at sensing tip 13 is less than 32 degrees C. or 89.6 degrees F., display 15 will indicate this condition as “---C” or “---F” and loop back to block 88. Thermometer 11 will automatically shut off after a relatively long time, such as about one minute, with no measured temperature reading higher than 32 degrees C. or 89.6 degrees F.

If the initial temperature measurement is equal to or above 32 degrees C. or 89.6 degrees F., the current temperature will be displayed on display 15 as shown in block 90. As also indicated in block 90, when thermometer 11 detects that the temperature is equal to or higher than 35.5 degrees C. or 96 degrees F. a melody will begin playing, provided the sound mode is on.

Once a melody begins playing, the thermometer 11 continues to take temperature measurements for about a time suitably long to insure accurate reading, typically about eight seconds, while the melody continues to play as shown in decision block 92. After this time period, such as about eight seconds, has passed in the melody mode, thermometer 11 gives a positive reinforcement or reward signal, such as an audible sound, for example by saying “great job”, “good work”, “excellent” and/or “thank you”, as generally shown in block 93. Thermometer 11 also stores the temperature reading and leaves the backlight activated for about a time adequate to read or record the temperature, such as for four seconds, as shown in block 93. As further shown in block 93, thermometer 11 will automatically shut off after a designated time of inactivity, such as in about one to five minutes, if the thermometer is not manually switched off during this time period.

If the sound mode is turned off, thermometer 11 will measure the temperature in the same manner as just described but without audible signaling, such as playing a melody, as shown in block 94. Once the measurement is complete, thermometer 11 will emit a signal, such as a short beep, display the temperature reading, store the temperature reading and leave the backlight activated for about the desired time, for example about four seconds, as shown in block 94. As shown in block 95, thermometer 11 will automatically shut off, such as in about one to five minutes, if not manually switched off.

If during the measuring cycle while the melody is playing, the temperature drops from its upward rise, this is understood as an indication that the sensing tip 13 is no longer properly positioned, and the melody stops playing. A temperature drop during not more than about 5 seconds will activate this feature. Typically this temperature drop is not more than about 4 seconds, not more than about 3 seconds, not more than about 2 seconds, or not more than about 1 second, depending on the sensitivity desired for a particular unit. When desired, the electronic circuitry includes a feature whereby the thermometer 11 gives an indication of non-compliance, such as by an audible statement, for example “try again”, as shown in block 96 after this temperature drop is detected.

The electronic circuitry can further include locking the temperature reading and either displaying the temperature or dashed lines with the backlight activated for about four seconds as shown in block 96. Also as shown in block 96, thermometer 11 will automatically shut off in a suitable time period, such as after about one minute if not manually switched off or restarted before then.

While preferred forms of the invention have been shown and described, it will be realized that alterations, modifications and/or combinations may be made thereto without departing from the scope of the following claims. For example, other process steps or data flow steps different from those illustrated in FIG. 6 can be practiced. Also, audible feedback other than music can be played to indicate proper positioning of the probe. In addition, audible cues other than cessation of music and a command to try again can be used when the probe ceases to be properly positioned.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. A digital thermometer for measuring the temperature of a living being by inserting a portion of the thermometer in the mouth of the being comprising:
a probe with first and second ends, and an intermediate portion disposed between the first and second ends;
a temperature sensor at the first end of the probe;
a housing portion at the second end of the probe;
said temperature sensor at the first end of the probe adapted to be inserted into the mouth and under the tongue of the being;
a display for displaying a temperature measured by said thermometer; and
electronic circuitry in communication with said temperature sensor to detect whether said probe is properly positioned, said electronic circuitry provides audible feedback indicating that said probe is properly positioned and detects a temperature drop that triggers audible feedback to signal that said probe ceases to be properly positioned.

2. The digital thermometer in accordance with claim 1, wherein said circuitry further provides audible direction to reposition said probe to restart temperature measurement after said audible feedback to signal that said probe ceases to be properly positioned.

3. The digital thermometer in accordance with claim 1, wherein said electronic circuitry temperature drop detection at said probe includes detection of a temperature decrease during a time period of not more than about 5 seconds.

4. The digital thermometer in accordance with claim 1, wherein said audible feedback for proper positioning is music.

5. The digital thermometer in accordance with claim 4 wherein the said music is a children's song.

6. The digital thermometer in accordance with claim 4, wherein said audible feedback when said probe becomes improperly positioned is to cease playing said music.

7. The digital thermometer in accordance with claim 1, wherein said audible direction when said probe ceases to be properly positioned comprises an audible command.

8. The digital thermometer in accordance with claim 1, wherein said housing comprises the shape of a children's character.

9. A digital thermometer for measuring the temperature of a living being by inserting a portion of the thermometer in the mouth of the being comprising:
a probe with first and second ends, and an intermediate portion disposed between the first and second ends;
a temperature sensor at the first end of the probe;
a housing portion at the second end of the probe;
said temperature sensor at the first end of the probe adapted to be inserted into the mouth and under the tongue of the being;
a display for displaying a temperature measured by said thermometer; and
electronic circuitry in communication with said temperature sensor to detect whether said probe is properly positioned, said electronic circuitry emits music as audible feedback that said probe is properly positioned and detects a cessation of temperature rise during a selected time period that triggers audible feedback comprising ceasing to emit music to thereby signal that said probe ceases to be properly positioned.

10. The digital thermometer in accordance with claim 9, wherein said electronic circuitry further provides audible direction to reposition said probe to restart temperature measurement after said audible feedback to signal that said probe ceases to be properly positioned.

11. The digital thermometer in accordance with claim 9, wherein said selected time period is not greater than about 5 seconds.

12. The digital thermometer in accordance with claim 9, wherein said selected time period is not greater than about 3 seconds.

13. The digital thermometer in accordance with claim 9, wherein said electronic circuitry further includes a signal emitter activated after temperature measurement proceeds for a given time period generally corresponding to time required for the temperature sensor to properly measure the body temperature of the being

14. The digital thermometer in accordance with claim 13, wherein said signal emitter comprises an audible signal.

15. A digital thermometer for measuring the temperature of a living being by inserting a portion of the thermometer in the mouth of the being comprising:
a probe with first and second ends, and an intermediate portion disposed between the first and second ends, said intermediate portion comprising a curved probe length;
a temperature sensor at the first end of the probe;
a housing portion at the second end of the probe, said housing portion being downwardly depending from the probe, and said temperature sensor being in temperature-measuring engagement with a surface within the mouth of the being when said probe of the thermometer is positioned within the mouth of the being;
a display for displaying a temperature measured by said thermometer; and
electronic circuitry in communication with said temperature sensor to detect whether said probe is properly positioned, said electronic circuitry provides audible feedback indicating that said probe is properly positioned and detects a temperature drop that triggers audible feedback to signal that said probe ceases to be properly positioned.

16. The digital thermometer in accordance with claim 15, wherein said circuitry further provides audible direction to reposition said probe to restart temperature measurement after said audible feedback to signal that said probe ceases to be properly positioned.

17. The digital thermometer in accordance with claim 15, wherein said housing comprises the shape of a children's character.

18. The digital thermometer in accordance with claim 15, wherein said electronic circuitry temperature drop detection at said probe includes detection of a rapid temperature decrease

19. The digital thermometer in accordance with claim 15, wherein said probe includes a grip member on said curved probe length, said grip member being positioned to engage the lip and/or teeth of the being

20. The digital thermometer in accordance with claim 19, including more than one grip member, and said grip members extend over a substantial part of said curved probe length.
